(19) United States
(12) Patent Application Publication
(10) Pub. No.: US 2017/0086751 A1
(43) Pub. Date: Mar. 30, 2017

(54) METATARSAL-PHALANGEAL JOINT STRENGTH MEASURING DEVICE

(71) Applicant: NIKE, Inc., Beaverton, OR (US)

(72) Inventors: Michael S. Amos, Beaverton, OR (US); Sharna M. Donovan, Hillsboro, OR (US); Thomas W. Jackson, Beaverton, OR (US); Gregory R. Leedy, Milwaukie, OR (US)

(73) Assignee: NIKE, Inc., Beaverton, OR (US)

(21) Appl. No.: 15/274,976

(22) Filed: Sep. 23, 2016

Related U.S. Application Data
(60) Provisional application No. 62/222,894, filed on Sep. 24, 2015.

(51) Int. Cl. A61B 5/00; A61B 5/22; A61B 5/11; A61B 5/103; A61B 5/107

(52) U.S. Cl. CPC A61B 5/702; A61B 5/1036; A61B 5/1074; A61B 5/1121; A61B 5/224; A61B 5/4528

(57) ABSTRACT

A metatarsal-phalangeal joint strength measuring device is provided and includes a base plate, a first toe plate, and a first force sensor associated with the first toe plate. The base plate receives a heel of a foot and defines a longitudinal axis extending between a first edge and a second edge. The first toe plate receives at least one toe of the foot, is movable relative to the base plate, and measures a load applied to the first toe plate.

### CROSS REFERENCE TO RELATED APPLICATIONS

This application claims priority to U.S. Provisional Application Ser. No. 62/222,894, filed Sep. 24, 2015, the disclosure of which is hereby incorporated by reference in its entirety.

### FIELD

The present disclosure relates generally to a device that measures strength applied across a metatarsal-phalangeal joint.

### BACKGROUND

This section provides background information related to the present disclosure which is not necessarily prior art.

An individual's metatarsal-phalangeal (MTP) joint strength can be used to select an appropriate article of footwear that attains desirable performance characteristics related to activities performed by the individual wearing the footwear. The individual's MTP joint strength is generally dependent on the extension of the toes of the foot. The toes may include a range of motion about the MTP joints of the foot allowing the toes to extend about an axis that extends through the MTP joints relative to a ground surface.

Extending the toes through the range of motion, causes muscles of the toes to elongate and be under tension. This tension acts as a resistance on the toes ability to apply force that increases as the toes extend further through the range of motion and, therefore, increases the tension of the toes. An individual's hallux, commonly referred to as the ‘big toe,’ may exhibit varying strengths through its range of motion that are different than varying strengths that the four lesser toes exhibit through their collective range of motion. For example, the hallux's ability to apply force may drastically decrease when an angular position through its range of motion is reached while, on the other hand, the four lesser toes' ability to apply force may not drastically decrease until the toes have reached a greater angular position through their range of motion. Accordingly, obtaining the MTP joint strength of the hallux at different angular positions in isolation from obtaining the MTP joint strength of the four lesser toes collectively at the different angular positions, can provide detailed information related to both the strength and flexibility of the hallux and the strength and flexibility of the four lesser toes that may be used to select an appropriate article of footwear for an individual.

### DRAWINGS

The drawings described herein are for illustrative purposes only of selected configurations and are not intended to limit the scope of the present disclosure.

FIGS. 1-3 are top perspective views of a metatarsal-phalangeal (MTP) joint strength measuring device in accordance with principles of the present disclosure;

FIG. 4 is a cross-sectional view of the MTP joint strength measuring device of FIG. 1 taken along line 4-4 showing an attachment feature slidably attaching a heel plate to a guide channel of a base plate;

FIGS. 5A-5D are schematic side views of the toe-strength measuring device of FIGS. 1-3 showing first and second toe plates pivotally moving about a first axis of rotation between a flat position (FIG. 5A) and a terminating angular position (FIG. 5D) when a foot is received by a base plate;

FIG. 6 is a front view of the MTP joint strength measuring device of FIG. 2 showing a first force sensor disposed between a first toe plate and a support plate and a second force sensor disposed between a second toe plate and the support plate;

FIG. 7 is a bottom perspective view of a support plate of the MTP joint measuring device of FIGS. 1-3 showing a receiving slot that receives a mounting member attached to a toe divider and a thru slot that receives a base portion of the toe divider; and

FIG. 8 is a top perspective view of the MTP joint strength measuring device showing a forefoot strap, a leg strap, and a mid-foot strap for securing a foot to the measuring device.

### DETAILED DESCRIPTION

Example configurations will now be described more fully with reference to the accompanying drawings. Example configurations are provided so that this disclosure will be thorough, and will fully convey the scope of the disclosure to those of ordinary skill in the art. Specific details are set forth such as examples of specific components, devices, and methods, to provide a thorough understanding of configurations of the present disclosure. It will be apparent to those of ordinary skill in the art that specific details need not be employed, that example configurations may be embodied in many different forms, and that the specific details and the example configurations should not be construed to limit the scope of the disclosure.

The terminology used herein is for the purpose of describing particular exemplary configurations only and is not intended to be limiting. As used herein, the singular articles “a,” “an,” and “the” may be intended to include the plural forms as well, unless the context clearly indicates otherwise. The terms “comprises,” “comprising,” “including,” and “having,” are inclusive and therefore specify the presence of features, steps, operations, elements, and/or components, but do not preclude the presence or addition of one or more other features, steps, operations, elements, components, and/or groups thereof. The method steps, processes, and operations described herein are not to be construed as necessarily requiring their performance in the particular order discussed or illustrated, unless specifically identified as an order of performance. Additional or alternative steps may be employed.

When an element or layer is referred to as being “on,” “engaged to,” “connected to,” “attached to,” or “coupled to” another element or layer, it may be directly on, engaged, connected, attached, or coupled to the other element or layer, or intervening elements or layers may be present. In contrast, when an element is referred to as being “directly on,” “directly engaged to,” “directly connected to,” “directly attached to,” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

The terms first, second, third, etc. may be used herein to describe various elements, components, regions, layers and/or sections. These elements, components, regions, layers and/or sections should not be limited by these terms. These terms may be only used to distinguish one element, component, region, layer or section from another region, layer or section. Terms such as “first,” “second,” and other numerical terms do not imply a sequence or order unless clearly indicated by the context. Thus, a first element, component, region, layer or section discussed below could be termed a second element, component, region, layer or section without departing from the teachings of the example configurations.

In one configuration, a metatarsal-phalangeal (MTP) joint strength measuring device is provided and includes a base plate, a first toe plate, and a first force sensor. The base plate receives a heel of a foot and defines a longitudinal axis that extends between a first edge and a second edge. The first toe plate receives at least one toe of the foot and is movable relative to the base plate. The first toe sensor is associated with the first toe plate and measures a load applied to the first toe plate.

In some implementations, the device includes a heel plate movably secured to the base plate and opposing an upper surface of the base plate. The heel plate may include a heel cup that receives the heel of the foot. In some examples, the heel plate is linearly movable relative to the base plate along the longitudinal axis. For instance, the heel plate may be slidably attached to a guide channel formed through an interior region of the base plate and extending substantially parallel to the longitudinal axis. Here, the guide channel may permit the heel plate to linearly move relative to the base plate along the longitudinal axis. In some examples, the heel plate is rotatable relative to the base plate. The heel plate may include a curved slot that guides rotational movement of the heel plate relative to the base plate. Optionally, the device includes a guide member opposing a bottom surface of the base plate that is disposed on an opposite side of the base plate than the upper surface. Here, the guide member may include a post that is received by the curved slot of the heel plate and movably secures the heel plate and the guide member to the base plate.

In some examples, the first toe plate includes a first edge disposed adjacent to the second edge of the base plate. The first toe plate may be pivotable relative to the base plate about a first axis of rotation substantially aligned with the first edge of the first toe plate. For example, the first toe plate may be pivotable relative to the base plate about the first axis of rotation between a flat position when the first toe plate is substantially coplanar with respect to the base plate and a terminating angular position when the first toe plate is disposed at an angle relative to the base plate. In this example, the slope of the first plate with respect to the base plate increases as the first toe plate pivotally moves toward the terminating angular position. In some examples, the device also includes a motor that moves the first toe plate relative to the base plate about the first axis of rotation. Additionally or alternatively, the device may include a rotational position sensor that measures the angular position of the first toe plate relative to the base plate.

In some implementations, the device also includes at least one wall extending from the base plate and pivotally supporting the first toe plate about the first axis of rotation. In some examples, the at least one wall receives a retaining member that retains the first toe plate at different angular positions relative to the base plate. The at least one wall may include apertures formed therethrough that receive the retaining member. For instance, the apertures may be positioned at different locations along the at least one wall to permit the first toe plate to be positioned at multiple angles relative to the base plate. Optionally, the at least one wall includes a slot formed therethrough that slidably receives the retaining member. Here, the retaining member may be selectively fixed along the length of the slot at various locations between a first end and a second end of the slot.

In some configurations, the first toe plate includes a first toe-engaging surface and a first contact surface disposed on an opposite side of the first toe plate than the first toe-engaging surface and opposing a support plate. The first toe-engaging surface may receive one of a hallux of the foot and the four lesser toes of the foot. The hallux is commonly referred to as the ‘big toe’ of the foot. In some examples, the first force sensor is disposed between the first contact surface and the support plate. The first force sensor may measure a load applied by the one of the hallux of the foot and the four lesser toes of the foot on the first toe-engaging surface. In some implementations, the device also includes a second toe plate disposed adjacent to the first toe plate and including a first edge that extends substantially co-linear with the first edge of the first toe plate. The second toe plate may be pivotally movable relative to the base plate about the first axis of rotation. In some configurations, the second toe plate includes a second toe-engaging surface and a second contact surface disposed on an opposite side of the second toe plate than the second toe-engaging surface and opposing the support plate. The second toe-engaging surface may receive the other one of the hallux of the foot and the four lesser toes of the foot. In some examples, the device also includes a second force sensor disposed between the second contact surface and the support plate. Here, the second force sensor may measure a load applied by the other one of the hallux of the foot and the four lesser toes of the foot on the second toe-engaging surface. The measured load applied to the second toe-engaging surface may be isolated from a load applied to the first toe-engaging surface.

In another aspect of the disclosure, a MTP joint strength measuring device is provided and includes a support plate and a first toe plate opposing the support plate for receiving a hallux of a foot. The MPT joint strength measuring device also includes a second toe plate that is substantially coplanar with the first toe plate, opposes the support plate, and receives the four lesser toes of the foot. The MTP joint strength measuring device also includes a first force sensor associated with the first toe plate and the support plate and a second force sensor associated with the second toe plate and the support plate. The first force sensor measures a load applied by the hallux to the first toe plate while the second force sensor measures a load applied by the four lesser toes to the second toe plate.

In some implementations, the support plate is movable relative to a ground surface in unison with the first toe plate and the second toe plate. Here, the first toe plate and the second toe plate may be pivotable about a first axis of rotation between multiple angular positions relative to the ground surface. The first axis of rotation may be substantially aligned with a first edge of the first toe plate and a first edge of the second toe plate.

In some examples, the MTP joint strength measuring device also includes a motor that pivotally moves the first toe plate and the second toe plate relative to the ground surface about the first axis of rotation. Additionally or alternatively, the MTP joint strength measuring device may include a rotational position sensor that measures the angular position of the first toe plate and the second toe plate relative to the ground surface.

In some configurations, the MTP joint strength measuring device also includes at least one wall pivotally supporting the support plate, the first toe plate, and the second toe plate about the first axis of rotation. The at least one wall may receive a retaining member that retains the first toe plate and the second toe plate at multiple angles relative to the ground surface. In some examples, the at least one wall includes apertures formed therethrough that receive the retaining member. The apertures may be positioned at different locations along the at least one wall to permit the first toe plate and the second toe plate to be positioned at the multiple angles relative to the ground surface. In some examples, the at least one wall includes a slot formed therethrough that slidably receives the retaining member. In these examples, the retaining member be selectively fixed along the length of the slot at various locations between a first end and a second end of the slot to permit the first toe plate and the second toe plate to be positioned at the multiple angles relative to the ground surface

In some implementations, the MTP joint strength measuring device includes a base plate that receives a heel of a foot and defines a longitudinal axis substantially parallel to a ground surface. The base plate may extend between a first edge and a second edge with one of the first edge and the second edge being disposed proximate to a first edge of the first toe plate and a first edge of the second toe plate. In some examples, a heel plate is movably secured to the base plate, opposes an upper surface of the heel plate, and includes a heel cup that receives the heel of the foot. The heel plate may be linearly movable relative to the base plate along the longitudinal axis. In some examples, the heel plate is slidably attached to a guide channel formed through an interior region of the base plate and extends substantially parallel to the longitudinal axis. Here, the guide channel may permit the heel plate to linearly move relative to the base plate along the longitudinal axis. In some implementations, the heel plate is rotatable relative to the base plate and may include a curved slot that guides rotational movement of the heel plate relative to the base plate. In some examples, the MTP joint strength measuring device also includes a guide member opposing a bottom surface of the base plate disposed on an opposite side of the base plate than the upper surface. In these examples, the guide channel may include a post that is received by the curved slot of the heel plate and movably secures the heel plate and the guide member to the base plate. In some configurations, at least one of the base plate and the heel plate includes at least one slot that receives a fastener to secure at least one of the foot to the base plate and the heel to the heel plate.

A method for measuring MTP joint strength is also provided and includes positioning a foot on a base plate defining a longitudinal axis that extends between a first edge and a second edge, aligning an anatomical feature of the foot with the second edge of the base plate, and positioning at least one of the toes on a first toe plate. The first toe plate is movable between multiple angular positions relative to the base plate and includes a first edge disposed adjacent to the second edge of the base plate. The method also includes measuring a load applied to the first toe plate.

In some implementations, aligning the anatomical feature of the foot includes aligning a bend line of all toes of the foot that extends through an axis of rotation of metatarsal-phalangeal joints of the foot. Positioning the foot on the base plate may include positioning a heel of the foot on a heel plate movably secured to the heel plate. The heel plate may include a heel cup that receives the heel of the foot.

In some scenarios, aligning the anatomical feature of the foot with the second edge of the base plate includes aligning the anatomical feature by linearly moving the heel plate relative to the base plate along the longitudinal axis. In these scenarios, linearly moving the heel plate relative to the base plate includes linearly moving the heel plate along a guide channel formed through an interior region of the base plate that extends substantially parallel to the longitudinal axis. The heel plate may be slidably attached to the guide channel. Additionally or alternatively, aligning the anatomical feature of the foot with the second edge of the base plate includes aligning the anatomical feature by rotating the heel plate relative to the base plate. In these scenarios, rotating the heel plate relative to the base plate includes guiding the heel plate along a curved slot formed through the heel plate to permit rotational movement of the heel plate relative to the base plate.

In some implementations, positioning at least one of the toes on a first toe plate includes positioning one of a hallux of the foot and four lesser toes of the foot on the first toe plate. Here, measuring a load applied to the first toe plate may include measuring a load applied by the one of the hallux and the four lesser toes to the first toe plate. In some examples, the method also includes positioning the other one of the hallux of the foot and the four lesser toes of the foot on a second toe plate disposed adjacent to the first toe plate. In these examples, the second toe plate is movable relative to the base plate and includes a first edge that extends substantially co-linear with the first edge of the first toe plate.

Optionally, the method also includes pivotally moving the first toe plate and the second toe plate about a first axis of rotation to permit the first toe plate and the second toe plate to be positioned at multiple angles relative to the base plate. Here, the first axis of rotation is substantially aligned with the first edge of the first toe plate and the first edge of the second toe plate. In some examples, the first toe plate and the second toe plate are selectively fixed at one of the different angles relative to the toe plate. An angular position of the first toe plate and the second toe plate relative to the base plate may be measured using a rotational position sensor.

In some implementations, measuring a load applied to the first toe plate includes measuring a load applied by the one of the hallux and the four lesser toes to the first toe plate using a first force sensor associated with the first toe plate. The measured load applied by the one of the hallux and the four lesser toes to the first toe plate may be isolated from a load applied by the other one of the hallux and the four lesser toes to the second toe plate. Additionally or alternatively, the method may also include measuring the load applied by the other one of the hallux and the four lesser toes to the second toe plate using a second force sensor associated with the second toe plate. The measured load applied by the other one of the hallux and the four lesser toes to the second toe plate may be isolated from the load applied to the first toe plate.

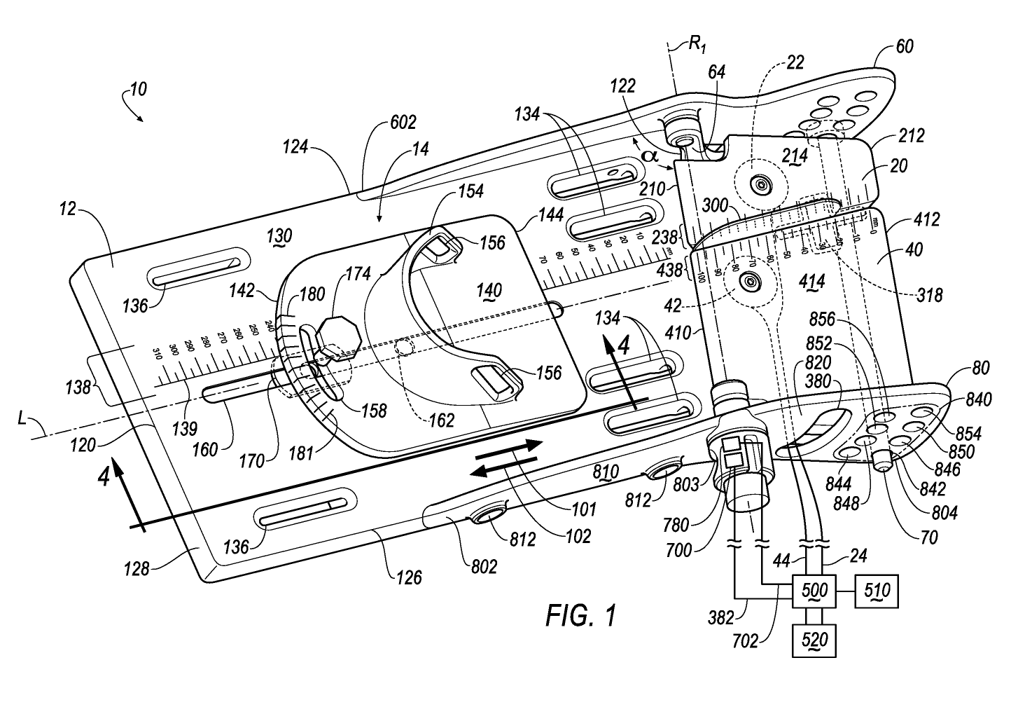

Referring to FIGS. 1-3, in some implementations, a metatarsal-phalangeal (MTP) joint strength measuring device 10 (hereinafter “measuring device 10”) includes a base plate 12 and first and second toe plates 20, 40, respectively, that are movable relative to the base plate 12. The first toe plate 20 may be associated with a first force sensor 22 that measures a load applied to the first toe plate 20. Similarly, the second toe plate 40 may be associated with a second force sensor 42 that measures a load applied to the second toe plate 40. The base plate 12 may receive a heel of a foot, the first toe plate 20 may receive a hallux of the foot, and the second toe plate 40 may receive the four lesser toes of the foot, as will be described in detail below.

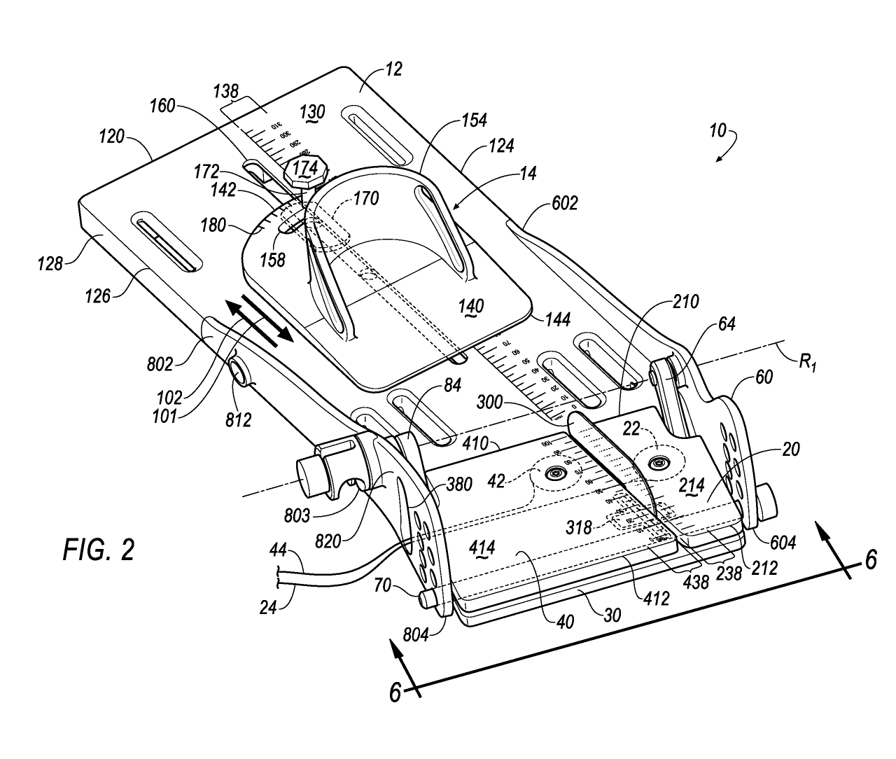

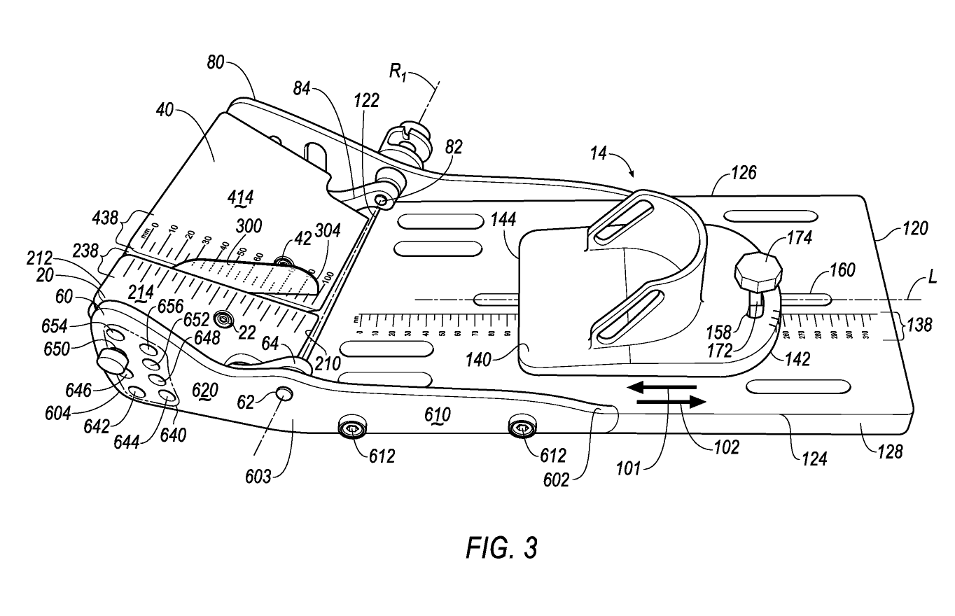

The measuring device 10 may also include a first wall 60 and a second wall 80 each extending from the base plate 12 and pivotally supporting the first and second toe plates 20, 40, respectively, about a first axis of rotation R₁. FIGS. 1 and 2 show the toe plates 20, 40 having an angular position of about 10 degrees relative to the base plate 12 and FIG. 3 shows the toe plates 20, 40 having an angular position of about 30 degrees relative to the base plate 12.

The base plate 12 may optionally include one or more retaining slots 134, 136 formed therethrough. For example, one or more forefoot retaining slots 134 may receive a fastener (e.g., a strap 956; FIG. 8) that wraps around a forefoot portion of a foot to secure the plantar surface of the forefoot on the base plate 12, thereby preventing the foot from moving while allowing the toes to move. Additionally or alternatively, one or more leg retaining slots 136 may receive a fastener (e.g., a strap 836; FIG. 8) that wraps over a knee or thigh associated with the foot to further assist in securing the foot on the base plate 12.

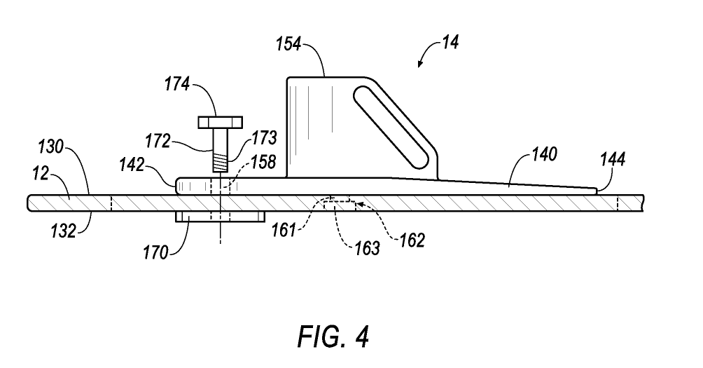

The base plate 12 includes an upper surface 130 and a bottom surface 132 (FIG. 4) disposed on an opposite side of the base plate 12 than the upper surface 130 and opposing the ground surface. A perimeter of the base plate 12 is defined by side edges 124, 126, a first edge 120, and a second edge 122. The base plate 12 may further define a longitudinal axis L that extends between the first edge 120 and the second edge 122 of the base plate 12. A flange 128 extends around the perimeter of the base plate 12 and may extend substantially perpendicular to the upper surface 130 and the lower surface 132. The flange 128 generally defines a thickness of the base plate 12 in a direction extending between the upper surface 130 and the lower surface 132.

The examples of FIGS. 1-3 show the side edge 124 corresponding to a medial side of a right foot and the side edge 126 corresponding to a lateral side of the right foot when the right foot is received on the base plate 12. Here, the side edge 124 has a length extending between the first edge 120 and the second edge 122 that is longer than a length of the side edge 126 extending between the first edge 120 and the second edge 122 on the opposite side of the base plate 12. Accordingly, the second edge 122 may be transverse to the first edge 120 of the base plate 12 to align an anatomical feature of the foot when the foot is received at the base plate 12. In some examples, the anatomical feature includes a bend line of all the toes of the foot that is received on the upper surface 130 of the base plate 12. The bend line may extend through the axis of rotation of the metatarsal-phalangeal (MTP) joints of the foot where proximal phalanges of the toes meet corresponding metatarsals of the foot. Accordingly, the bend line may define a pivot axis where the toes are permitted to extend through a range of motion about the MTP joints that includes different angular positions of the toes relative to the base plate 12 and the ground surface. In some examples, the pivot axis of the toes aligns with the first axis of rotation R₁.

In some implementations, a heel assembly 14 is disposed on the upper surface 130 of the base plate 12. The heel assembly 14 may include a heel plate 140 movably secured to the base plate 12 and opposing the upper surface 130 of the base plate 12. The heel plate 140 may include a heel cup 154 that receives the heel of the foot when the foot is received by the measuring device 10. The heel cup 154 may be fixedly secured to the heel plate 140 by a fastener or, alternatively, the heel cup 154 may be integrally formed with the heel plate 140. The heel plate 140 may define a longitudinal axis that extends between a first edge 142 and a second edge 144. The second edge 144 may be aligned to pass thru the arch of the foot when the foot is received on the heel plate 140. In some examples, the heel plate 140 tapers toward the upper surface 130 from the first edge 142 to the second edge 144. In some examples, the heel plate 140 is linearly movable relative to the base plate 12 along the along the longitudinal axis L of the base plate 12. Additionally or alternatively, the heel plate 140 may be rotatable relative to the base plate 12. One or more retaining slots 156 may be formed through the heel cup 154. The mid-foot retaining slots 156 may receive a fastener (e.g., a strap; FIG. 8) that wraps over the mid-foot portion of the foot, around the ankle, and back over the mid-foot portion of the foot to secure the foot to the heel plate 140.

In some configurations, the heel plate 140 is slidably attached to a guide channel 160 formed through an interior portion of the base plate 12 that extends substantially parallel to the longitudinal axis L of the base plate 12. For instance, the heel assembly 14 may include an attachment feature 162 having a pin 161 fixedly attached to the heel plate 140 at one end and a roller or bearing 163 (FIG. 4) disposed at the other end that is slidably received by the guide channel 160. For instance, the roller or bearing 163 of the attachment feature 162 may facilitate movement of the pin 161 relative to and within grooves (not shown) defined by the guide channel 160. Referring to FIG. 4, a cross-sectional view taken along line 4-4 of FIG. 1 shows the attachment feature 162 slidably attaching the heel plate 140 to the guide channel 160. Here, the guide channel 160 may permit the heel plate 140 to linearly move relative to the base plate 12 along the longitudinal axis when the guide channel 160 receives the roller or bearing 163 of the attachment feature 162. Accordingly, FIGS. 1-3 show that the heel plate 140 may linearly move relative to the base plate 12 along the longitudinal axis L in a first (forward) direction 101 toward the second edge 122 of the base plate 12 and in an opposite, second (rearward) direction 102 toward the first edge 120 of the base plate 12 so that the anatomical feature of the foot (e.g., the bend line through the axis of rotation of the MTP joints) is aligned with the second edge 122 of the base plate 12 regardless of the length of the particular foot. In some examples, the upper surface 130 of the base plate 12 includes a foot length gauge 138 that may be used to determine a position of the heel plate 140 relative to the base plate 12. Namely, the gauge 138 may include a series of graduations 139 that may be aligned with one or both edges 142, 144 of the heel plate 140 to determine the position of the heel plate 140 relative to the base plate 12.

FIGS. 1-4 show the heel plate 140 including a curved slot 158 located proximate to the first edge 142 of the heel plate 140. The curved slot 158 may guide rotational movement of the heel plate 140 relative to the base plate 12 and about the attachment feature 162 that slidably attaches the heel plate 140 to the guide channel 160. Namely, the curved slot 158 may receive a post 172 that cooperates with the slot 158 to guide rotational movement of the heel plate 140 relative to the base plate 12.

The post 172 is fixed for movement with the heel plate 140 and may be fixed at various locations between a first end and a second end of the curved slot 158 so that the heel plate 140 may be positioned at multiple angular positions relative to the base plate 12 and about the attachment feature 162. Accordingly, the heel plate 140 may be rotated to a desired angular position relative to the base plate 12 so that the anatomical feature of the foot (e.g., the bend line through the axis of rotation of the MTP joints) is aligned with the second edge 122 of the base plate 12. In so doing, the longitudinal axis of the heel plate 140 may shift between being substantially parallel to the longitudinal axis L of the base plate 12 and being transposed at different angular positions relative to the longitudinal axis L of the base plate 12 and about the attachment feature 162. In some examples, the heel plate 140 includes a foot angle gauge 180 that identifies an angle of the heel plate 140 relative to the base plate 12. Specifically, the foot angle gauge 180 may indicate an angle of the longitudinal axis of the heel plate 140 relative to the longitudinal axis L of the base plate 12. The angle may be determined by aligning graduation marks 181 of the gauge 180 with a feature of the base plate 12. For example, the graduation marks 181 may be aligned with an edge of the guide channel 160.

Aligning the anatomical feature of the foot with the second edge 122 of the base plate 12 may include at least one of linearly moving the heel plate 140 relative to the base plate 12 and rotating the heel plate 140 relative to the base plate 12. The heel assembly 14 may include a guide member 170 that may be adjusted to permit the linear and rotatable movement of the heel plate 140 relative to the base plate 12. Conversely, the guide member 170 may also be adjusted to prevent linear and rotatable movement of the heel plate 140 relative to the base plate 12 by securing the heel plate 140 to the base plate 12. FIG. 4 shows the guide member 170 opposing the bottom surface 132 of the base plate 12 within a gap between the bottom surface 132 and the ground surface.

The guide member 170 receives the post 172 extending through the curved slot 158 of the heel plate 140 and the guide channel 160 of the base plate 12 in a direction substantially perpendicular to the longitudinal axis L of the base plate 12. One end of the post 172 may be secured to an adjustment knob 174 for common rotation and the other end may be releasably fastened to the guide member 170. For clarity, FIGS. 1-4 show the post 172 and knob 174 removed from the curved slot 158, the guide channel 160, and the guide member 170. Here, the post 172 and the guide member 170 may include corresponding threads 173 to secure the post 172 to the guide member 170 and/or the knob 174.

In scenarios when linear and/or rotatable movement of the heel plate 140 relative to the base plate 12 is desirable so that an anatomical feature of the foot can be aligned with the second edge 122 of the base plate 12, the adjustment knob 174 may be turned in one direction (e.g., counter-clockwise) to loosen the post 172 from the guide member 170. In so doing, the effective length of the post 172—between the guide member 170 and the knob 174—is increased. Increasing the effective length of the post 172 releases engagement between the guide member 170 and the bottom surface 132 of the base plate 112 and releases engagement between the adjustment knob 174 and the heel plate 140, thereby allowing the guide member 170 and the heel plate 140 to move (linearly and rotationally) relative to the base plate 12. Conversely, in scenarios when it is desirable to prevent both linear and rotatable movement of the heel plate 140 relative to the base plate 12, such as when the anatomical feature of the foot is properly aligned with the second edge 122 of the base plate 12, the adjustment knob 174 may be turned in an opposite direction (e.g., clockwise). Turning the adjustment knob 174 in the clockwise direction reduces the effective length of the post 172—between the guide member 170 and the adjustment knob 174. In so doing, the guide member 170 cooperates with the adjustment knob 174 to compress the base plate 12 and the heel plate 140 therebetween.

The compressive force applied to the base plate 12 and the heel plate 140 by the guide member 170 and the adjustment knob 174 restricts relative movement between these components which, in turn, secures a position of the heel plate 140 relative the guide plate 12. Specifically, as the guide member 170 secures to the bottom surface 132 of the base plate 12, the heel plate 140 attached thereto is compressed between the adjustment knob 174 and the upper surface 130, thereby securing the heel plate 140 to the upper surface 130 of the base plate 12 so that both the guide member 170 and the heel plate 140 are prevented from moving (linearly and rotationally) relative to the base plate 12. In some examples, friction between the guide member 170 and the heel plate 140 on the bottom surface 132 and the upper surface 130, respectively, is sufficient to prevent the guide member 170 and the heel plate 140 attached thereto from freely moving relative to the base plate 12. Additionally or alternatively, the bottom surface 132 and/or the upper surface 130 may include retention members extending therefrom that retain the guide member 170 and/or the heel plate 140 to the base plate 12 so that relative movement is prevented.

In some implementations, the first toe plate 20 includes a first edge 210 disposed adjacent to the second edge 122 of the base plate 12. The first edge 210 of the first toe plate 20 may be substantially parallel to the second edge 122 of the base plate 12. The first toe plate 20 may have a length extending from the first edge 210 to a second edge 212 that extends away from the base plate 12 and is substantially parallel to the first edge 210. As shown in FIG. 1, the second edge 212 of the base plate 12 and the first edge 210 of the first toe plate 20 are positioned at an angle α relative to the side edge 124 of the base plate 12 that is less than ninety degrees) (90°. Accordingly, the first toe plate 20 extends away from the side edge 124 in a direction from the first edge 210 to the second edge 212.

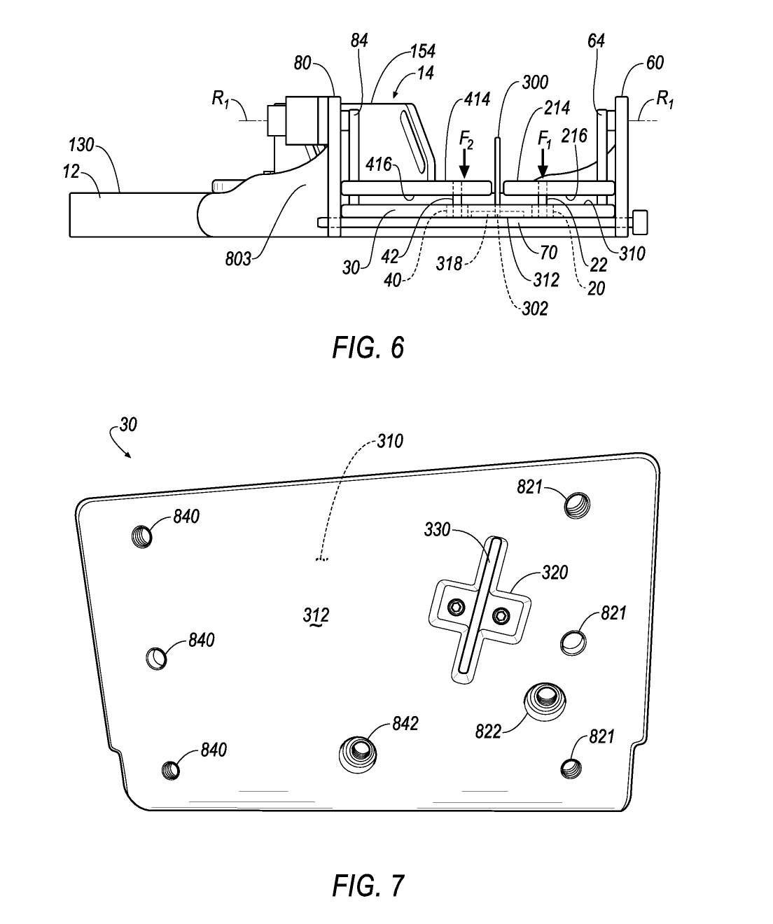

A first toe-engaging surface 214 of the first toe plate 20 may extend between the first edge 210 and the second edge 212 and may be positioned to receive at least one toe of a foot. For example, the first toe-engaging surface 214 may receive the hallux of a foot when the foot is received at the base plate 12 and the anatomical feature (e.g., bend line through the axis of rotation of the MTP joints) is aligned with the second edge 122 of the base plate 12. The first toe plate 20 may also include a first contact surface 216 (FIG. 6) disposed on an opposite side of the first toe plate 20 than the first toe-engaging surface 214 and opposing a support plate 30. The first toe plate 20 and the support plate 30 may be pivotally movable in unison relative to the base plate 12 about the first axis of rotation R₁ substantially aligned with the first edge 210 of the first toe plate 20, as will be described in detail below. In some examples, the first toe-engaging surface 214 includes a hallux length gauge 238 having graduations that may be used to determine a length of the hallux of the foot when the foot is received at the base plate 12 and the anatomical feature is aligned with the second edge 122 of the base plate 12.

The second toe plate 40 may be disposed adjacent to the first toe plate 20 and may include a first edge 410 disposed adjacent to the second edge 122 of the base plate 12 and a second edge 412 disposed on an opposite end of the second toe plate 40 than the first edge 410. The first edge 410 may be substantially co-linear with the first edge 210 of the first toe plate 20 such that the first edge 410 is similarly disposed at an angle α (less than ninety degrees)(90°) relative to the side edge 124. As such, the first edge 410 of the second toe plate 40 may be substantially parallel to the second edge 122 of the base plate 12. While the first toe plate 20 is disposed adjacent to the second toe plate 40, the second toe plate 40 may be spaced apart from the first toe plate 20 in a direction extending substantially parallel to the first edges 210, 410, as shown in FIGS. 1 and 6. The second edge 412 may likewise be substantially co-linear with the second edge 212 of the first toe plate 20.

As with the first toe plate 20, the second toe plate 40 may extend in a direction away from the side edge 124 between the first edge 410 and the second edge 412. The first edge 410 and the second edge 412 may be substantially parallel to one another and to the second edge 122 of the base plate 12. Accordingly, the second toe plate 40 angles outward from the side edge 126 of the base plate 12 corresponding to the lateral side of the right foot.

A second toe-engaging surface 414 of the second toe plate 40 extends between the first edge 410 and the second edge 412 and is positioned to receive at least one other toe of a foot when the foot is received by the measurement device 10. For example, the second toe-engaging surface 414 may receive the four lesser toes of the foot (e.g., right foot) when the foot is received by the base plate 12 and the anatomical feature (e.g., bend line through the axis of rotation of the MTP joints) is aligned with the second edge 122 of the base plate 12. The second toe plate 40 may also include a second contact surface 416 (FIG. 6) disposed on an opposite side of the second toe plate 40 than the second toe-engaging surface 414 and opposing the support plate 30. The second toe plate 40, the first toe plate 20, and the support plate 30 may be pivotally movable in unison relative to the base plate 12 about the first axis of rotation R₁, which is substantially aligned with the first edges 210, 410 of the first and second toe plates 20, 40, respectively. In some examples, the second toe-engaging surface 414 includes a lesser toes length gauge 438 having graduations that may be used to determine a length of the lesser toes of the foot when the foot is received at the base plate 12 and the anatomical feature is aligned with the second edge 122 of the base plate 12.

In some implementations, at least one of the first wall 60 and the second wall 80 extend from the base plate 12 and pivotally support the first toe plate 20, the second toe plate 40, and the support plate 30, about the first axis of rotation R₁. A first end 602 of the first wall 60 may be disposed proximate to the medial side edge 124 of the base plate 12 between the first and second edges 120, 122, respectively, while a second end 604 of the first wall 60 may be disposed proximate to the second edge 212 of the first toe plate 20. The first wall 60 may slant inwardly from the medial side edge 124 at a bending region 603 located proximate to the first axis of rotation R₁ between the first end 602 and the second end 604. In some examples, the first wall 60 is divided into a first portion 610 located between the first end 602 and the bending region 603 and a second portion 620 located between the bending region 603 and the second end 604. For instance, the first portion 610 may extend along a portion of the perimeter flange 128 at the medial side edge 124 in a direction substantially parallel to the longitudinal axis L of the base plate 12 and the second portion 620 may extend away from the second edge 122 of the base plate 12 in a direction substantially perpendicular to the second edge 122 at approximately the same angle α, as described above with respect to the first toe plate 20 and the second toe plate 40.

In these examples, the first wall 60 includes a top edge that increasingly extends away from the upper surface 130 of the base plate 12 between the first end 602 and the bending region 603 (FIG. 3). Accordingly, the first portion 610 defines a height at the bending region 603 that is greater than a height of the top edge at the first end 602. The top edge of the first wall 60 may also increase in height from the bending region 603 to the second end 604 such that the first wall 60 is at its highest point at the second end 604. Thus, the second portion 620 may define a height at the second end 604 that is greater than the height of the top edge at the bending region 603 and at the first end 602.

The first portion 610 may be attached to the perimeter flange 128 using one or more fasteners 612 or, alternatively, may be integrally formed therewith. For example, the base plate 12 and the first wall 60 may be machined out of a block of aluminum or steel.

A first end 802 of the second wall 80 may be disposed proximate to the lateral side edge 126 of the base plate 12 between the first and second edges 120, 122, respectively, while a second end 804 of the second wall 80 may be disposed proximate to the second edge 412 of the second toe plate 40. The second wall 80 may slant outwardly from the lateral side edge 126 at a bending region 803 located proximate to the first axis of rotation R₁ between the first and second ends 802, 804, respectively. In some examples, the second wall 80 is divided into a first portion 810 located between the first end 802 and the bending region 803 and a second portion 820 located between the bending region 803 and the second end 804. For instance, the first portion 810 may extend along a portion of the perimeter flange 128 at the lateral side edge 126 in a direction substantially parallel to the longitudinal axis L of the base plate 12 and the second portion 820 may extend away from the second edge 122 of the base plate 12 in a direction substantially perpendicular to the second edge 122 and substantially parallel to the second portion 620 of the first wall 60.

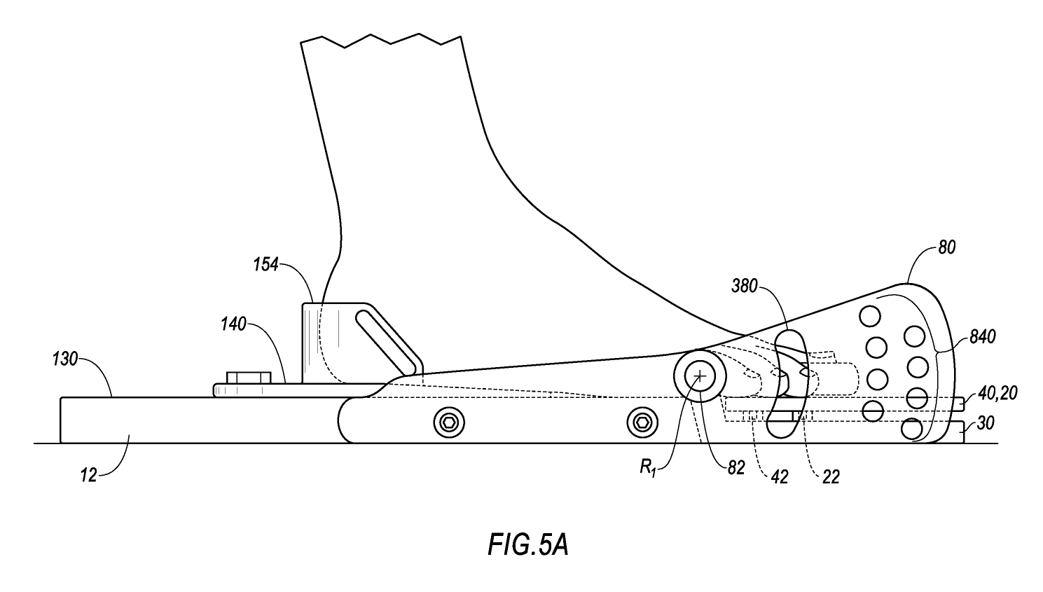

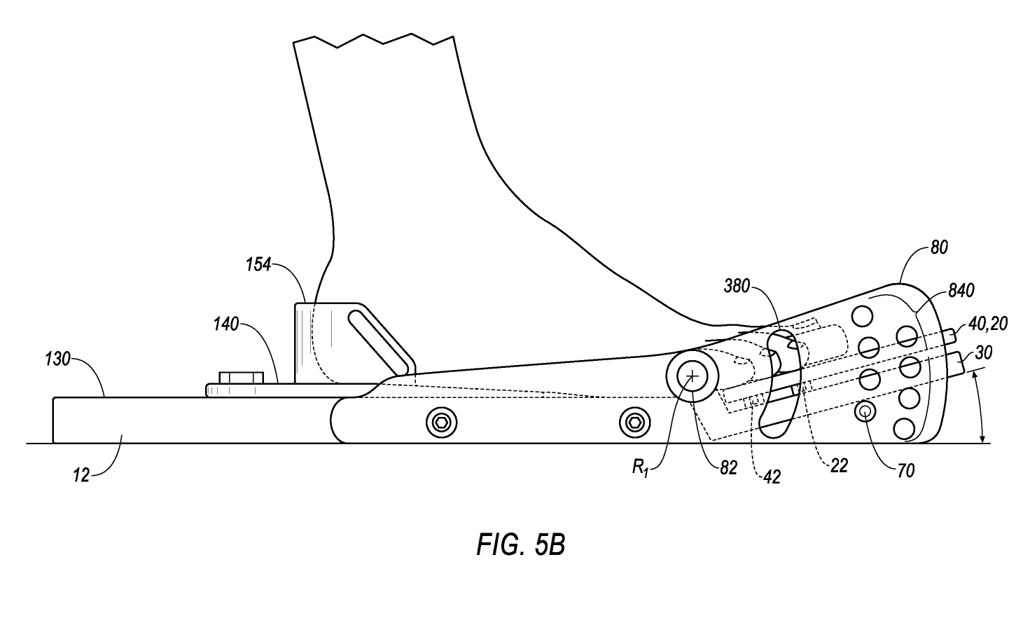

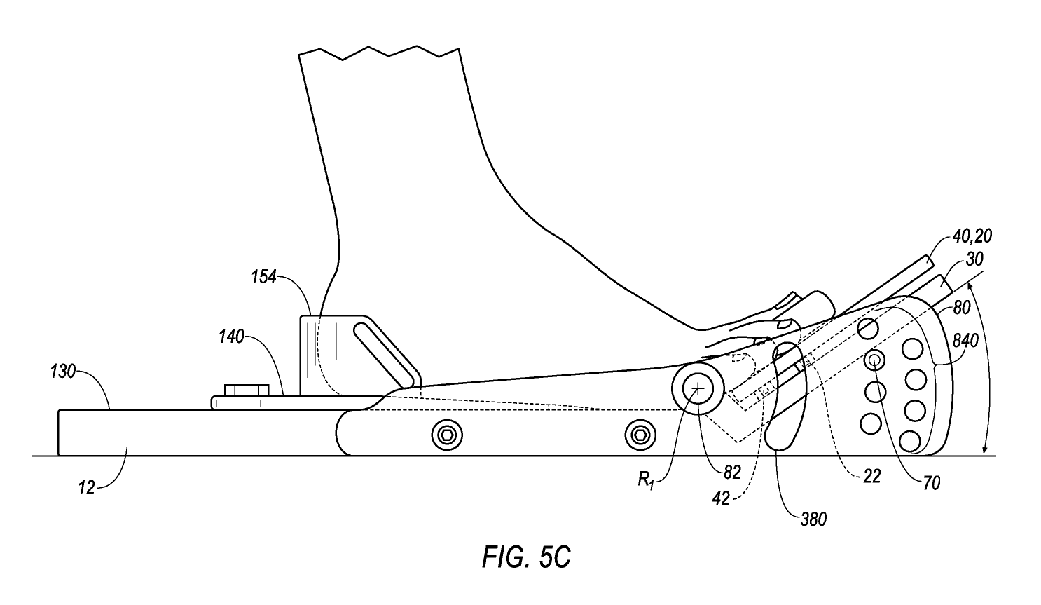

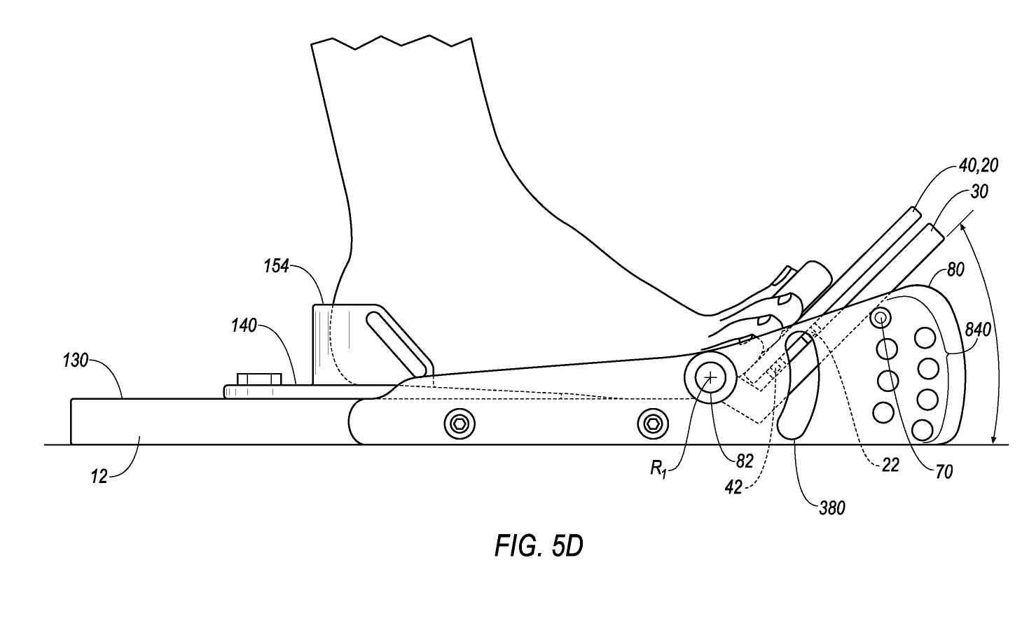

In these examples, the second wall 80 includes a top edge that increasingly extends away from the upper surface 130 of the base plate 12 between the first end 802 and the bending region 803 (FIG. 5A). Accordingly, the first portion 810 defines a height at the bending region 803 that is greater than a height of the top edge at the first end 802. The top edge of the second wall 80 may also increase in height from the bending region 803 to the second end 804 such that the second wall 80 is at its highest point at the second end 804. Thus, the second portion 820 may define a height at the second end 804 that is greater than the height of the top edge at the bending region 803 and at the first end 802.

The first portion 810 may be attached to the perimeter flange 128 using one or more fasteners 812 or, alternatively, may be integrally formed therewith. For example, the base plate 12 and the second wall 80 may be machined out of a block of aluminum or steel, as described above with respect to the first wall 60.

At least one of the first wall 60 and the second wall 80 may pivotally support the first toe plate 20 and the second toe plate 40 about the first axis of rotation R₁. For instance, the first wall 60 may pivotally support the first toe plate 20 and the second toe plate 40 about the first axis of rotation R₁ at a pivot point 62. Additionally or alternatively, the second wall 80 may pivotally support the first toe plate 20 and the second toe plate 40 about the first axis of rotation R₁ at a pivot point 82. Accordingly, the first axis of rotation R₁ may be substantially aligned with the first edges 210, 410, of the first and second toe plates 20, 40, respectively, so that toes received on the toe plates 20, 40 may flex through a range of motion as the toe plates 20, 40 pivotally move about the first axis of rotation R₁ relative to the base plate 12.

In some configurations, a first pivot arm 64 extends from the pivot point 62 of the first wall 60 to pivotally support at least one of the support plate 30 and the first toe plate 20 relative to the first wall 60 and a second pivot arm 84 extends from the pivot point 82 of the second wall 80 to pivotally support at least one of the support plate 30 and the second toe plate 40 relative to the second wall 80. The pivot arms 64, 84 may include proximal ends rotatably coupled to their respective pivot points 62, 82 and distal ends attached to at least one of the support plate 30, the first toe plate 20, and the second toe plate 40. Namely, the pivot arm 64 may include a proximal end coupled to the pivot point 62 and a distal end coupled to at least one of the support plate 30 and the first toe plate 20 while the pivot arm 84 may include a proximal end coupled to the pivot point 82 and a distal end coupled to at least one of the support plate 30 and the second toe plate 40. In some implementations, the pivot points 62, 82 may be located at a greater height from the ground surface than the toe plates 20, 40 to allow the toe plates 20, 40 (and also the support plate 30) to pivotally move relative the base plate 12 without interference from the ground surface.

The first toe plate 20 and the second toe plate 40 may be pivotally movable relative to the base plate 12 about the first axis of rotation R₁ between a flat position (FIG. 5A) and a terminating angular position (FIG. 5D). The flat position corresponds to the first and second toe plates 20, 40, respectively, being substantially coplanar with respect to the base plate 12 (e.g., the upper surface 130). The terminating angular position corresponds to the first and second toe plates 20, 40, respectively, being disposed at an angle relative to the base plate 12. In some examples, the terminating angular position of the toe plates 20, 40 relative to the base plate 12 includes an angle of at least 45 degrees (45°). The toe plates 20, 40 may be disposed at any angular position between the flat position and the terminating angular position. For example, a slope of the toe plates 20, 40 with respect to the base plate 12 may increase as the toe plates 20, 40 pivotally move from the flat position toward the terminating angular position. In operation, the first force sensor 22 may measure a load applied by the hallux of the foot to the first toe-engaging surface 214 and the second force sensor 42 may measure a load applied by the four lesser toes of the foot collectively to the second toe-engaging surface 414 when the first and second toe plates 20, 40, respectively, are positioned at different angles relative to the base plate 12.

Referring to FIGS. 1-3, in some implementations, at least one of the walls 60, 80 of the measuring device 10 receives a retaining member 70 that retains the toe plates 20, 40 at different angular positions relative to the base plate 12. For example, the support plate 30 may rest on the retaining member 70 so that the toe plates 20, 40 are retained at a desired angular position relative to the base plate 12. In some examples, more than one retaining members 70 may be used to retain the toe plates 20, 40 at the different angular positions.

In some examples, the first wall 60 includes a series of apertures 640 (FIG. 3) formed therethrough that are sized to receive the retaining member 70. Additionally or alternatively, the second wall 80 may include a corresponding series of apertures 840 (FIG. 1) formed therethrough that are sized to receive the retaining member 70. The series of apertures 640, 840 may be positioned at different locations along their respective second portions 620, 820 of the walls 60, 80 to permit the first and second toe plates 20, 40, respectively, to be fixed at multiple different angles relative to the base plate 12. In this way, each aperture 640, 840 may correspond to a respective angular position for retaining the toe plates 20, 40 when the retaining member 70 is received therein. In examples, when more than one retaining member 70 is used, each retaining 70 is received within the respective ones of the apertures associated with the desired angular position for retaining the toe plates 20, 40.

For example, FIG. 3 shows the series of apertures 640 formed through the first wall 60 including an aperture 642 corresponding to an angle of 10 degrees, an aperture 644 corresponding to an angle 15 degrees, an aperture 646 corresponding to an angle of 20 degrees, an aperture 648 corresponding to an angle of 25 degrees, an aperture 650 corresponding to an angle of 30 degrees, an aperture 652 corresponding to an angle of 35 degrees, an aperture 654 corresponding to an angle of 40 degrees, and an aperture 656 corresponding to an angle of 45 degrees. FIG. 3 shows the aperture 650 receiving the retaining member 70 to retain the toe plates 20, 40 at an angle of 30 degrees relative to the base plate 12. Likewise, FIG. 1 shows the series of apertures 840 formed through the second wall 80 including apertures 842, 844, 846, 848, 850, 852, 854, 856 that correspond to angles of 10, 15, 20, 25, 30, 35, 40, and 45 degrees, respectively. The apertures 642, 644, 646, 648, 650, 652, 654, 656 are respectively coaxially aligned with the apertures 842, 844, 846, 848, 850, 852, 854, 856 such than when the retaining member 70 is disposed in aperture 644, the retaining member 70 is likewise disposed in aperture 844, for example.

FIG. 1 shows the aperture 842 receiving the retaining member 70 to retain the toe plates 20, 40 at an angle of 10 degrees relative to the base plate 12. Namely, the retaining member 70 engages the support plate 30 to maintain the support plate 30 and, thus, the toe plates 20, 40 in a desired angled position relative to the base plate 12. When the retaining member 70 is not received by any of the apertures 640, 840, the toe plates 20, 40 will be disposed in the flat position (e.g., FIG. 5A) substantially coplanar with the base plate 12.

Referring to FIG. 1, in some implementations, the measuring device optionally includes a servo motor 700 that pivotally moves the toe plates 20, 40 relative to the base plate 12 about the first axis of rotation R₁. For example, the motor 700 may be rotatably coupled to the pivot point 62 of the first wall 60 (or the pivot point 82 of the second wall 80) to pivotally move the toe plates 20, 40 relative to the base plate 12. In some implementations, the measuring device 10 includes a computing device 500 in communication with the servo motor 700 via a signal path 382 that may be wired or wireless to allow the computing device 500 to variably control the servo motor 700 to rotate the toe plates 20, 40 to different angular positions relative to the base plate 12.

In some configurations, the measuring device 10 includes a rotational position sensor 780 that measures the angular position of the toe plates 20, 40 relative to the base plate 12. In some examples, the rotational position sensor 780 measures rotation about at least one of the pivot points 62, 82. The rotational position sensor 780 may be in communication with the servo motor 700 and may measures rotation of the servo motor 700 to determine the angular position of the toe plates 20, 40. In some configurations, the rotational position sensor 780 communicates the measured angular positions to the computing device 500 via a signal path 702 that may be wired or wireless. The computing device 500 may also receive load data collected by the first and second force sensors 22, 42, respectively. For example, the first force sensor 22 may provide measured loads applied to the first toe plate 20 to the computing device 500 via the first wire 24 and the second force sensor 42 may provide measured loads applied to the second toe plate 40 to the computing device 500 via the second wire 44. The wires 24, 44 may extend through a slot 380 formed through the wall 80. The slot 380 is positioned and sized to allow the wires 24, 44 to move relative to the wall 80 throughout the full range of motion of the support plate 30 from the configuration shown in FIG. 5A to the configuration shown in FIG. 5D.

In some examples, the computing device 500 is in communication with non-transitory memory 520 that stores the load data collected by the force sensors 22, 42 and/or the angular position of the toe plates 20, 40 measured by the rotational position sensor 780. The computing device 500 may be associated with a display 510 that displays the load data and/or the angular position data.

Referring to FIG. 5A-5D, a schematic side view of the measuring device 10 of FIG. 1 shown from the lateral side 126 of the base plate 12 shows the first toe plate 20 and the second toe plate 40 pivotally movable about the first axis of rotation R₁ between the flat position and the terminating angular position when a right foot is received by the base plate 12 (e.g., on the heel plate 140) and the anatomical feature (e.g., bend line through the axis of rotation of the MTP joints) aligned with the second edge 122 of the base plate 12. The first toe plate 20 is substantially coplanar with the second toe plate 40 and, therefore, the first toe plate 20 is obstructed by the second toe plate 40 in the views of FIGS. 5A-5D. The support plate 30 opposing the toe plates 20, 40 is also movable relative to the base plate 12 (and the ground surface) in unison with the toe plates 20, 40, respectively. In some examples, a user manually moves the toe plates 20, 40 to the different angular positions relative to the base plate 12 by applying a force on at least one of the support plate 30, the first toe plate 20, and the second toe plate 40. In other examples, the servo motor 700 (FIG. 1) moves the plates 20, 40 to the different angular positions relative to the base plate 12 by applying a force on the pivot 82 and/or pivot arm 84 to change a position of the support plate 30 and, thus, the first toe plate 20 and/or the second toe plate 40 relative to the base plate 12.

FIG. 5A shows the first and second toe plates 20, 40, respectively, in the flat position being substantially coplanar with respect to the base plate 12. Here, the toes are in a neutral state and not being extended.

FIG. 5B shows the toe plates 20, 40 disposed at an angle relative to the base plate 12. The angle of the toe plates 20, 40 relative to the base plate 12 includes an angular position between the flat position and the terminating angular position. The angle of the toe plates 20, 40 may be substantially 15 degrees in FIG. 5B so that the toes are slightly extended about their range of motion.

Referring to FIG. 5C, the toe plates 20, 40 are disposed at an angle relative to the base plate 12 that is greater than the angle of the toe plates 20, 40 in FIG. 5B. The angle of the toe plates 20, 40 in FIG. 5C may include an angular position between the flat position and the terminating angular position. For example, the angle of the toe plates 20, 40 may be substantially 35 degrees in FIG. 5C so that the toes are extended to a greater degree than when the toe plates 20, 40 are disposed at the angle of substantially 15 degrees shown in FIG. 5B.

FIG. 5D shows the toe plates 20, 40 disposed at an angle relative to the base plate 12 that corresponds to the terminating angular position. In some examples, the terminating angular position is 45 degrees. Here, the toes of the foot are extended further about their range of motion compared to the extension of the toes when the toe plates 20, 40 are disposed at angles of substantially 15 degrees (FIG. 5B) and 35 degrees (FIG. 5C), respectively.

In some configurations, the first force sensor 22 associated with the first toe plate 20 is disposed between the first contact surface 216 and the support plate 30 and the second force sensor 42 associated with the second toe plate 40 is disposed between the second contact surface 416 and the support plate 30. The first force sensor 22 and the second force sensor 42 may also be embedded within their respective toe plate 20, 40. Referring to FIG. 6, a front view taken from the perspective of line 6-6 of FIG. 2 shows the first force sensor 22 disposed between the first contact surface 216 and the support plate 30 and the second force sensor 42 disposed between the second contact surface 416 and the support plate 30. The first and second toe plates 20, 40, respectively, each measure a load applied to their respective toe plates 20, 40 that is isolated from a load applied to the other toe plate 20, 40.

FIG. 6 shows a first load F₁ (e.g., a first force vector) applied to the first toe-engaging surface 214 of the first toe plate 20 that is measurable by the first force sensor 22. In some examples, the hallux of the right foot applies the first load F₁ to the first toe-engaging surface 214 and the first force sensor 22 measures the first load F₁ applied by the hallux independent of any loading applied by one or more of the remaining toes received on the second toe-engaging surface 414 of the second toe plate 40. FIG. 6 also shows a second load F₂ (e.g., a second force vector) applied to the second toe-engaging surface 414 of the second toe plate 40 that is measured by the second force sensor 42. In some examples, the four lesser toes of the foot collectively apply the second load F₂ to the second toe-engaging surface 414 and the second force sensor 42 measures the second load F₂ applied by the four lesser toes independent of the first load F₁ applied by the hallux received on the first toe-engaging surface 214.

In some scenarios, the first force sensor 22 may measure the first load F₁ applied to the first toe-engaging surface 214 by the hallux at different angular positions of the first toe plate 20 relative to the base plate 12. Likewise, the second force sensor 42 may measure the second load F₂ applied to the second toe-engaging surface 414 of the second toe plate 40 by the four lesser toes at different angular positions of the second toe plate 40 relative to the base plate 12. In some examples, the force sensors 22, 42 are embedded within their associated plates 20, 40, and also within the support plate 30. At least one of the force sensors 22, 42 may include a conventional load-cell that measures the total force between the associated toe plate(s) 20, 40 and the support plate 30. Optionally, at least one of the force sensors 22, 42 may include an absolute pressure sensor securely mounted to the support plate 30 so that a pressure distribution may be measured when a load (e.g., force vector(s) F₁, F₂) is applied to the associated toe plate(s) 20, 40. The present disclosure is not limited to any one particular type of force sensor 22, 42, and may employ any suitable type of force sensor to measure loads applied by the toes of the foot to the first and second toe plates 20, 40, respectively.

In some configurations, each of the force sensors 22, 42 may transmit respective electrical signals indicative of load data that corresponds to the applied load measurements F₁, F₂ to the computing device 500 (FIG. 1) in communication with each of the force sensors 22, 42. The computing device 500 may execute signal processing electronics to determine the values of the applied load measurements F₁, F₂. In some examples, the first wire 24 provides the communication between the first force sensor 22 and the computing device 500, while the second wire 44 provides the communication between the second force sensor 42 and the computing device 500. In other examples, the force sensors 22, 42 may communicate wirelessly with the computing device 500. The computing device 500 may display collected load data from each of the force measurements F₁, F₂ on the display 510 (FIG. 1) in communication with the computing device 500. Additionally or alternatively, the computing device 500 may provide the collected load data to other computing devices over a network. The computing device 500 may also store the load measurements F₁, F₂ in non-transitory memory 520 (FIG. 1) in communication with the computing device 500.

In some implementations, referring to FIG. 6, the support plate 30 includes a bottom surface 312 and an upper surface 310 disposed on an opposite side of the support plate 30 than the bottom surface 312. As shown in FIG. 6, the upper surface 310 opposes and is spaced apart from the first and second contact surfaces 216, 416, respectively, of the toe plates 20, 40.

In some examples, a toe divider 300 extends from the upper surface 310 of the support plate 30 and through the gap separating the first toe plate 20 and the second toe plate 40. As such, a portion of the toe divider extends through the gap in a direction away from and substantially perpendicular to the toe-engaging surfaces 214, 414. The portion of the toe divider 300 that extends through the gap may terminate at a distal end 304 (FIG. 3) located proximate to the first edges 210, 410 of the toe plates 20, 40. The toe divider 300 may serve to separate the hallux from the other or lesser toes when the foot is received by the measuring device 10. Namely, the divider 300 may be used to position the hallux relative to the first toe plate 20 and to position the other toes relative to the second toe plate 40. For example, the toe divider 300 may position the hallux such that the hallux opposes the first toe-engaging surface 214 of the first toe plate 20. In so doing, the divider 300 prevents the hallux from contacting any portion of the second toe plate 40. Similarly, the divider 300 may position the four lesser toes relative to the second toe-engaging surface 414 of the second toe plate 40 so that none of the four lesser toes contact any portion of the first toe plate 20. Positioning the toes in the foregoing manner ensures that the forces measured by the first force sensor 22 are only attributed to the hallux and the forces measured by the second force sensor 42 are only attributed to the four other toes, collectively. This way, an accurate measurement of MTP joint strength can be determined for the hallux separately from the other toes and vice versa.

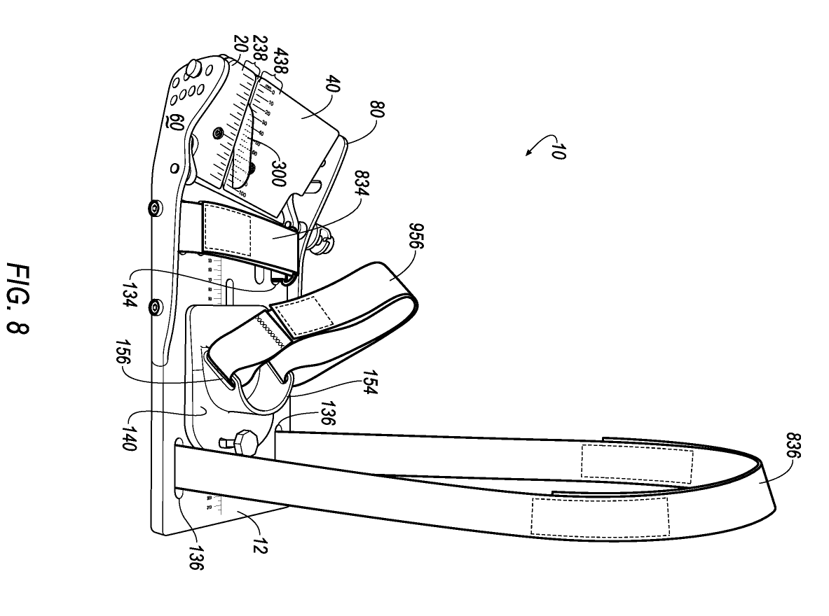

In some examples, the toe divider 300 is removably attached to the support plate 30. For example, FIG. 6 shows a base portion 302 of the toe divider 300 attached to a mounting member 318 that is releasably secured to the support plate 30. Referring to FIG. 7, a bottom perspective view of the support plate 30 shows a receiving slot 320 formed in the bottom surface 312 of the support plate 30 and a thru slot 330 that extends through the support plate 30. The receiving slot 320 may include a size and shape that matingly receives the mounting member 318 attached to the base portion 302 of the toe divider 300. For example, the receiving slot 320 may include one or more threaded holes for receiving fasteners (none shown) when the mounting member 318 is received therein. The thru slot 330 may be sized and shaped to receive the base portion 302 of the toe divider 300 and may be aligned with the gap separating the first toe plate 20 and the second toe plate 40.

With continued reference to FIG. 7, the support plate 30 is shown as including one or more apertures 821 formed therethrough for securing the first toe plate 20 to the support plate 30 and one or more apertures 840 formed therethrough for securing the second toe plate 40 to the support plate 30. The support plate 30 may additionally include apertures 822, 842 formed through the support plate 30 that respectively receive a portion of the first force sensor 22 and the second force sensor 42 to support the sensors 22, 42 between the support plate 30 and respective ones of the first toe plate 20 and the second toe plate 40. Supporting the sensors 22, 42 between the support plate 30 and the respective toe plates 20, 40 allows the sensors 22, 42 to measure pressure exerted on the toe plates 20, 40 by the toes of a user when the user's foot is disposed in the measuring device 10.

Referring to FIG. 8, a top perspective view of the measuring device 10 shows an optional forefoot strap 834 releasably fastened to the forefoot retaining slots 134 formed in the base plate 12. The forefoot strap 834 may be adjustable to wrap over the forefoot of the foot for securing the plantar surface of the foot to the base plate 12, thereby restricting the foot from moving while permitting the toes of the foot to flex about their range of motion as the toe plates 20, 40 pivotally move about the first axis of rotation R₁ relative to the base plate 12. Optionally, a leg strap 836 may be releasably fastened to the leg retaining slots 136 formed in the base plate 12. The leg strap 836 may be adjustable to wrap over the knee or thigh associated with the foot for securing the plantar surface of the foot to the base plate 12. The leg strap 836 may be used to wrap over the knee or thigh while the individual associated with the foot is in a seated position so that body weight does not influence data collected by the measuring device 10. Additionally or alternatively, a mid-foot strap 956 may be releasably fastened to the mid-foot retaining slots 156 formed in the heel cup for securing the heel of the foot to the heel plate 140. The straps 834, 836, and 956 may be used in any combination or not used at all. Further, each strap 834, 836, 956 may include a fastening mechanism such as a hook-and-loop fastener that allows a length of each strap 834, 836, 956 to be adjusted in an effort to accommodate feet and legs having different lengths and sizes. In short, the straps 834, 836, 956 provide the measuring device 10 with a degree of adjustability to allow the device 10 to be used with a wide range of individuals. This adjustability, when taken in conjunction with the linear and angular adjustability of the heel assembly 14 relative to the base plate 12 allows the measuring device 10 to be used with virtually any individual, as the rotational axis of the individual's toes can be aligned with the first axis of rotation R₁ by adjusting the position of the heel assembly 14 relative to the base plate 12 and by adjusting the straps 834, 836, 956. While the straps 834, 836, 956 do not adjust a position of a foot relative to the base plate 12 per se, the straps 834, 836, 956 may need to be adjusted to permit proper movement of the heel assembly 14 relative to the base plate 12 (i.e., to provide clearance for a foot when the heel assembly 14 is adjusted relative to the base plate 12) and, further, to ensure proper engagement with the foot to restrict movement of the foot and leg of the individual relative to the base plate 12 when MTP joint strength measurements are taken.

The measuring device 10 may be used to measure MTP joint strength at different angular positions through the range of motion of the toes. In some implementations, MTP joint strength is measured dynamically by measuring respective loads applied to the toe plates 20, 40 as the toe plates 20, 40 pivotally move about the first axis of rotation R₁. Specifically, when the heel of the foot is received at the heel plate 140 and the anatomical feature (e.g., bend line through the axis of rotation of the MTP joints) is aligned with the second edge 122 of the base plate 12 and the first axis of rotation R₁, the toes are received on the toe plates 20, 40 in a neutral position when the toe plates 20, 40 are in the flat position (FIG. 5A).

In operation, the toe plates 20, 40 may pivotally move about the first axis of rotation R₁ away from the flat position by exerting a force on the support plate 30. Movement of the support plate 30 from the position shown in FIG. 5A toward the positions shown in FIGS. 5B-5D causes the force applied to the support plate 30 to similarly be applied to the toes at the first toe plate 20 and the second toe plate 40. In so doing, the first force sensor 22 and the second force sensor 42 measure the load applied to their respective toe-engaging surfaces 214, 414 as the toes increasingly extend through their range of motion when the angular position of the toe plates 20, 40 relative to the base plate 12 increases. Thus, the loads measured by the first force sensor 22 correspond to a resistance applied by the hallux against the first toe-engaging surface 214, and the loads measured by the second force sensor 42 correspond to a resistance applied by the four lesser toes against the second toe-engaging surface 414.

Generally, the toes will provide their greatest passive resistance when their flexibility limit is reached and, therefore, the range of motion of the toes can be correlated with the angular position of the toe plates 20, 40 relative to the base plate 12 and the applied loads (toe resistance) measured by each of the force sensors 22, 42. For instance, a flexibility limit or range of motion of the hallux may be determined based on the angular position of the first toe plate 20 relative to the base plate 12 when the first force sensor 22 measures a threshold passive resistance applied to the first toe-engaging surface 214. Likewise, a flexibility limit or range of motion of the four lesser toes may be determined based on the angular position of the second toe plate 40 relative to the base plate 12 when the second force sensor 42 measures a threshold passive resistance applied to the second toe-engaging surface 414.

In some examples, the computing device 500 may receive the applied load measurements from each of the force sensors 22, 42 at each of the angular positions measured by the rotational position sensor 780. Specifically, the force sensors 22, 42 may provide signals associated with each of the angular positions measured by the rotational position sensor 780 to the computing device 500, and the computing device 500 may compute load measurements based on the received signals. Measuring the MTP joint strength at different ranges of motion can be used to aid a person in selecting an appropriate article of footwear for a particular need or training regimen. For example, an article of footwear may be matched with the individual based on the strength and flexibility data collected by the measuring device 10. Moreover, the strength and flexibility data collected may be used to tailor specific exercises for improving strength and flexibility of the toes.

As described, a force may be applied to the support plate 30 to move the support plate 30 from the position shown in FIG. 5A toward the positions shown in FIGS. 5B-5D. The applied force may be manually applied in an effort to flex the toes about their range of motion. For example, an operator user may lift the support plate 30 or the toe plates 20, 40 to cause movement of the support plate 30 and, thus, the first toe plate 20 and the second toe plate 40, relative to the base plate 12. While the applied force is described as being manually applied, the servo motor 700 or other force-generating device may be used to move the toe plates 20, 40 relative to the base plate 12 about the first axis of rotation R₁. For example, the servo motor 700 may apply a rotational force on the support plate 30 and, thus, the first toe plate 20 and the second toe plate 40, to rotate the plates 20, 30, 40 from the position shown in FIG. 5A toward the positions shown in FIGS. 5B-5D.

In a second mode of operation, MTP joint strength is measured statically by measuring respective loads applied to the toe plates 20, 40 as the toe plates 20, 40 are fixed at different angular positions with respect to the base plate 12. For example, when the heel of the foot is received at the heel plate 140 and the anatomical feature (e.g., bend line through the axis of rotation of the MTP joints) is aligned with the second edge 122 of the base plate 12 and the first axis of rotation R₁, the toe plates 20, 40 may be fixed at a desired angular position with respect to the base plate 12 to measure the strength of the toes of the foot. For example, the first force sensor 22 may measure a force applied by the hallux pushing against the first toe-engaging surface 214, and the second force sensor 42 may measure a force applied by the four lesser toes collectively pushing against the second toe-engaging surface 414. The first and second force sensors 22, 42, respectively, may measure forces applied to the respective toe plates 20, 40 while the toe plates 20, 40 are fixed at different angular positions with respect to the base plate 12. Namely, the retaining member 70 may be received by the apertures 642, 842 to retain the toe plates 20, 40 at an angle of 10 degrees with respect to the base plate 12 while the force sensors 22, 42 may measure the forces applied by the toes pushing against the respective toe-engaging surfaces 214, 414. The retaining member 70 may be removed from apertures 642, 842 and received by other corresponding pairs of the series of apertures 640, 840 to retain the toe plates 20, 40 at different angular positions to measure the associated forces applied thereto by the toes of the foot. Accordingly, correlations between MTP joint strength and flexibility can be determined for both the hallux and the four lesser toes in isolation while all of the toes are extended at different angular positions about their range of motion.

By retaining the toe plates at a selected angular position relative to the base plate 12, the strength of the toes can be measured at different angular positions of the toe plates 20, 40 relative to the base plate 12. Namely, load data (exerted toe force) may be measured by each of the force sensors 22, 42 at a plurality of angular positions of the toe plates 20, 40 relative to the base plate 12. Measuring the MTP joint strength at different angles can be used to aid a person in selecting an appropriate article of footwear for a particular need or training regimen. For example, an article of footwear may be matched with the individual based on the strength and flexibility data collected by the measuring device 10. Moreover, the strength and flexibility data collected may be used to tailor specific exercises for improving strength and flexibility of the toes.

While the examples depict a MTP joint strength measuring device 10 for measuring MTP joint strength and flexibility of a right foot, a similar MTP joint measuring device can be utilized for measuring toe strength and flexibility of a left foot without departing from the present disclosure. Moreover, while the examples include the walls 60, 80 interconnecting the base plate 12 and the support plate 30 to form the measuring device 10 as a single unitary member, the walls 60, 80 may be omitted in other configurations, thereby separating the base plate 12 and the support plate 30. In some implementations, the base plate 12 may be omitted and the foot may rest upon the ground surface.

The following Clauses provide an exemplary configuration for the MTP joint strength measuring device 10 described above (Clauses 1-38) or a method of measuring metatarsal-phalangeal (MTP) joint strength (Clauses 39-53).

Clause 1: A metatarsal-phalangeal (MTP) joint strength measuring device comprising a base plate operable to receive a heel of a foot, the base plate defining a longitudinal axis extending between a first edge and a second edge a first toe plate operable to receive at least one toe of the foot, the first toe plate movable relative to the base plate and a first force sensor associated with the first toe plate and operable to measure a load applied to the first toe plate.

Clause 2: The device of Clause 1, further comprising a heel plate movably secured to the base plate and opposing an upper surface of the base plate, the heel plate including a heel cup operable to receive the heel of the foot.

Clause 3: The device of Clause 2, wherein the heel plate is linearly movable relative to the base plate along the longitudinal axis.

Clause 4: The device of Clause 3, wherein the heel plate is a slidably attached to a guide channel formed through an interior region of the base plate and extends substantially parallel to the longitudinal axis, the guide channel operable to permit the heel plate to linearly move relative to the base plate along the longitudinal axis.

Clause 5: The device of Clause 2, wherein the heel plate is rotatable relative to the base plate.

Clause 6: The device of Clause 5, wherein the heel plate includes a curved slot that guides rotational movement of the heel plate relative to the base plate.

Clause 7: The device of Clause 6, further comprising a guide member opposing a bottom surface of the base plate disposed on an opposite side of the base plate than the upper surface, the guide member including a post that is received by the curved slot of the heel plate and operable to movably secure the heel plate and the guide member to the base plate.

Clause 8: The device of any of the preceding Clauses, wherein the first toe plate includes a first edge disposed adjacent to the second edge of the base plate, the first toe plate pivotally movable relative to the base plate about a first axis of rotation substantially aligned with the first edge of the first toe plate.

Clause 9: The device of Clause 8, wherein the first toe plate is pivotally movable relative to the base plate about the first axis of rotation between a flat position when the first toe plate is substantially coplanar with respect to the base plate and a terminating angular position when the first toe plate is disposed at an angle relative to the base plate.

Clause 10: The device of Clause 9, wherein a slope of the first toe plate with respect to the base plate increases as the first toe plate pivotally moves toward the terminating angular position.

Clause 11: The device of any of the preceding Clauses, further comprising a motor operable to pivotally move the first toe plate relative to the base plate about the first axis of rotation.

Clause 12: The device of any of the preceding Clauses, further comprising a rotational position sensor operable to measure the angular position of the first toe plate relative to the base plate.

Clause 13: The device of any of the preceding Clauses, further comprising at least one wall extending from the base plate and pivotally supporting the first toe plate about the first axis of rotation.

Clause 14: The device of Clause 13, wherein the at least one wall receives a retaining member operable to retain the first toe plate at different angular positions relative to said base plate.

Clause 15: The device of Clause 14, wherein the at least one wall includes a series of apertures formed therethrough and operable to receive the retaining member, the series of apertures being positioned at different locations along said at least one wall to permit the first toe plate to be positioned at multiple angles relative to the base plate.

Clause 16: The device of Clause 14, wherein the at least one wall includes a slot formed therethrough and operable to slidably receive the retaining member, the retaining member operable to be selectively fixed along the length of the slot at various locations between a first end and a second end of the slot.

Clause 17: The device of any of the preceding Clauses, wherein the first toe plate includes a first toe-engaging surface and a first contact surface disposed on an opposite side of the first toe plate than the first toe-engaging surface and opposing a support plate, the first toe-engaging surface operable to receive one of a hallux of the foot and the four lesser toes of the foot.

Clause 18: The device of Clause 17, wherein the first force sensor is disposed between the first contact surface and the support plate, the first force sensor operable to measure a load applied by the one of the hallux of the foot and the four lesser toes of the foot to the first toe-engaging surface.

Clause 19: The device of Clause 17, further comprising a second toe plate disposed adjacent to the first toe plate and including a first edge that extends substantially co-linear with the first edge of the first toe plate, the second toe plate pivotally movable relative to the base plate about the first axis of rotation.

Clause 20: The device of Clause 19, wherein the second toe plate includes a second toe-engaging surface and a second contact surface disposed on an opposite side of the second toe plate than the second toe-engaging surface and opposing the support plate, the second toe-engaging surface operable to receive the other one of the hallux of the foot and the four lesser toes of the foot.

Clause 21: The device of Clause 20, further comprising a second force sensor disposed between the second contact surface and the support plate, the second force sensor operable to measure a load applied by the other one of the hallux of the foot and the four lesser toes of the foot to the second toe-engaging surface, the measured load applied to the second toe-engaging surface isolated from a load applied to the first toe-engaging surface.

Clause 22: A metatarsal-phalangeal (MTP) joint strength measuring device comprising:

a support plate and a first toe plate opposing the support plate and operable to receive a hallux of a foot and a second toe plate coplanar with the first toe plate and opposing the support plate, the second toe plate operable to receive the four lesser toes of the foot and a first force sensor associated with the first toe plate and the support plate, the first force sensor operative to measure a load applied by the hallux to the first toe plate and a second force sensor associated with the second toe plate and the support plate, the second force sensor operative to measure a load applied by the four lesser toes to the second toe plate.

Clause 23: The device of Clause 22, wherein the support plate is movable relative to a ground surface and in unison with the first toe plate and the second toe plate.

Clause 24: The device of any of Clauses 22-23, wherein the first toe plate and the second toe plate are pivotally movable about a first axis of rotation between multiple angular positions relative to the ground surface, the first axis of rotation substantially aligned with a first edge of the first toe plate and a first edge of the second toe plate.

Clause 25: The device of any of Clauses 22-24, further comprising a motor operable to pivotally move the first toe plate and the second toe plate relative to the ground surface about the first axis of rotation.

Clause 26: The device of any of Clauses 22-25, further comprising a rotational position sensor operable to measure the angular position of the first toe plate and the second toe plate relative to the ground surface.

Clause 27: The device of any of Clauses 22-26, further comprising at least one wall pivotally supporting the support plate, first toe plate, and the second toe plate about the first axis of rotation.

Clause 28: The device of Clause 27, wherein the at least one wall receives a retaining member operable to retain the first toe plate and the second toe plate at multiple angles relative to the ground surface.

Clause 29: The device of Clause 28, wherein the at least one wall includes a series of apertures formed therethrough and operable to receive the retaining member, the series of apertures being positioned at different locations along the at least one wall to permit the first toe plate and the second toe plate to be positioned at the multiple angles relative to the ground surface.

Clause 30: The device of Clause 28, wherein the at least one wall includes a slot formed therethrough and operable to slidably receive the retaining member, the retaining member operable to be selectively fixed along the length of the slot at various locations between a first end and a second end of the slot to permit the first toe plate and the second toe plate to be positioned at the multiple angles relative to the ground surface.

Clause 31: The device of any of Clauses 22-30, further comprising a base plate operable to receive a heel of a foot and defining a longitudinal axis substantially parallel to a ground surface, the base plate extends between a first edge and a second edge with one of the first edge and the second edge being disposed proximate to a first edge of the first toe plate and a first edge of the second toe plate.

Clause 32: The device of Clause 31, further comprising a heel plate movably secured to the base plate and opposing an upper surface of the base plate, the heel plate including a heel cup operable to receive the heel of the foot.

Clause 33: The device of Clause 32, wherein the heel plate is linearly movable relative to the base plate along the longitudinal axis.

Clause 34: The device of Clause 32, wherein the heel plate is slidably attached to a guide channel formed through an interior region of the base plate and extends substantially parallel to the longitudinal axis, the guide channel operable to permit the heel plate to linearly move relative to the base plate along the longitudinal axis.

Clause 35: The device of Clause 32, wherein the heel plate is rotatable relative to the base plate.

Clause 36: The device of Clause 35, wherein the heel plate includes a curved slot operable to guide rotational movement of the heel plate relative to the base plate.

Clause 37: The device of Clause 36, further comprising a guide member opposing a bottom surface of the base plate disposed on an opposite side of the base plate than the upper surface, the guide member including a post that is received by the curved slot of the heel plate and operable to movably secure the heel plate and the guide member to the base plate.

Clause 38: The device of Clause 32, wherein at least one of the base plate and the heel plate includes at least one slot operable to receive a fastener to secure at least one of the foot to the base plate and the heel to the heel plate.

Clause 39: A method for measuring metatarsal-phalangeal (MTP) joint strength comprising positioning a foot on a base plate defining a longitudinal axis that extends between a first edge and a second edge and aligning an anatomical feature of the foot with the second edge of the base plate and positioning at least one of the toes on a first toe plate, the first toe plate movable between multiple angular positions relative to the base plate and including a first edge disposed adjacent to the second edge of the base plate and measuring a load applied to the first toe plate.

Clause 40: The method of Clause 39, wherein aligning the anatomical feature of the foot includes aligning a bend line of all toes of the foot that extends through metatarsal-phalangeal joints of the foot.

Clause 41: The method of any of Clauses 39-40, wherein positioning the foot on the base plate includes positioning a heel of the foot on a heel plate movably secured to the base plate, the heel plate including a heel cup operable to receive the heel of the foot.

Clause 42: The method of any of Clauses 39-41, wherein aligning the anatomical feature of the foot with the second edge of the base plate includes aligning the anatomical feature by linearly moving the heel plate relative to the base plate along the longitudinal axis.

Clause 43: The method of Clause 42, wherein linearly moving the heel plate relative to the base plate includes linearly moving the heel plate along a guide channel formed through an interior region of the base plate that extends substantially parallel to the longitudinal axis, the heel plate slidably attached to the guide channel.

Clause 44: The method of any of Clauses 39-43, wherein aligning the anatomical feature of the foot with the second edge of the base plate includes aligning the anatomical feature by rotating the heel plate relative to the base plate.

Clause 45: The method of Clause 44, wherein rotating the heel plate relative to the base plate includes guiding the heel plate along a curved slot formed through the heel plate to permit rotational movement of the heel plate relative to the base plate.

Clause 46: The method of any of Clauses 39-45, wherein positioning at least one of the toes on a first toe plate includes positioning one of a hallux of the foot and four lesser toes of the foot on the first toe plate.

Clause 47: The method of Clause 46, wherein measuring a load applied to the first toe plate includes measuring a load applied by the one of the hallux and the four lesser toes to the first toe plate.

Clause 48: The method of Clause 46, further comprising positioning the other one of the hallux of the foot and the four lesser toes of the foot on a second toe plate disposed adjacent to the first toe plate, the second toe plate movable relative to the base plate and including a first edge that extends substantially co-linear with the first edge of the first toe plate.

Clause 49: The method of Clause 48, further comprising pivotally moving the first toe plate and the second toe plate about a first axis of rotation to permit the first toe plate and the second toe plate to be positioned at multiple angles relative to the base plate, the first axis of rotation substantially aligned with the first edge of the first toe plate and the first edge of the second toe plate.

Clause 50: The method of Clause 49, further comprising selectively fixing the first toe plate and the second toe plate at one of the different angles relative to the base plate.

Clause 51: The method of Clause 49, further comprising measuring an angular position of the first toe plate and the second toe plate relative to the base plate using a rotational position sensor.

Clause 52: The method of Clause 48, wherein measuring a load applied to the first toe plate includes measuring a load applied by the one of the hallux and the four lesser toes to the first toe plate using a first force sensor associated with the first toe plate, the measured load applied by the one of the hallux and the four lesser toes to the first toe plate isolated from a load applied by the other one of the hallux and the four lesser toes to the second toe plate.

Clause 53: The method of Clause 52, further comprising measuring the load applied by the other one of the hallux and the four lesser toes to the second toe plate using a second force sensor associated with the second toe plate, the measured load applied by the other one of the hallux and the four lesser toes to the second toe plate isolated from the load applied to the first toe plate.

The foregoing description has been provided for purposes of illustration and description. It is not intended to be exhaustive or to limit the disclosure. Individual elements or features of a particular configuration are generally not limited to that particular configuration, but, where applicable, are interchangeable and can be used in a selected configuration, even if not specifically shown or described. The same may also be varied in many ways. Such variations are not to be regarded as a departure from the disclosure, and all such modifications are intended to be included within the scope of the disclosure.

What is claimed is:

1. A metatarsal-phalangeal (MTP) joint strength measuring device comprising:
a base plate operable to receive a heel of a foot, the base plate defining a longitudinal axis extending between a first edge and a second edge;
a first toe plate operable to receive at least one toe of the foot, the first toe plate movable relative to the base plate; and
a first force sensor associated with the first toe plate and operable to measure a load applied to the first toe plate.

2. The device of claim 1, further comprising a heel plate movably secured to the base plate and opposing an upper surface of the base plate, the heel plate including a heel cup operable to receive the heel of the foot.

3. The device of claim 2, wherein the heel plate is linearly movable relative to the base plate along the longitudinal axis.

4. The device of claim 2, wherein the heel plate is rotatable relative to the base plate.

5. The device of claim 1, further comprising a motor operable to pivotally move the first toe plate relative to the base plate about the first axis of rotation.

6. The device of claim 1, further comprising a rotational position sensor operable to measure an angular position of the first toe plate relative to the base plate.

7. The device of claim 1, further comprising at least one wall extending from the base plate and pivotally supporting the first toe plate about the first axis of rotation, the at least one wall receiving a retaining member operable to retain the first toe plate at different angular positions relative to the base plate.

8. The device of claim 1, wherein the first toe plate includes a first toe-engaging surface and a first contact surface disposed on an opposite side of the first toe plate than the first toe-engaging surface and opposing a support plate, the first toe-engaging surface operable to receive one of a hallux of the foot and the four lesser toes of the foot.

9. The device of claim 8, wherein the first force sensor is disposed between the first contact surface and the support plate, the first force sensor operable to measure a load applied by the one of the hallux of the foot and the four lesser toes of the foot to the first toe-engaging surface.

10. The device of claim 8, further comprising a second toe plate disposed adjacent to the first toe plate and including a first edge that extends substantially co-linear with the first edge of the first toe plate, the second toe plate pivotally movable relative to the base plate about the first axis of rotation and including a second toe-engaging surface and a second contact surface disposed on an opposite side of the second toe plate than the second toe-engaging surface and opposing the support plate, the second toe-engaging surface operable to receive the other one of the hallux of the foot and the four lesser toes of the foot.

11. The device of claim 10, further comprising a second force sensor disposed between the second contact surface and the support plate, the second force sensor operable to measure a load applied by the other one of the hallux of the foot and the four lesser toes of the foot to the second toe-engaging surface, the measured load applied to the second toe-engaging surface isolated from a load applied to the first toe-engaging surface.

12. A metatarsal-phalangeal (MTP) joint strength measuring device comprising:
a support plate;
a first toe plate opposing the support plate and operable to receive a hallux of a foot;
a second toe plate coplanar with the first toe plate and opposing the support plate, the second toe plate operable to receive the four lesser toes of the foot;
a first force sensor associated with the first toe plate and the support plate, the first force sensor operative to measure a load applied by the hallux to the first toe plate; and
a second force sensor associated with the second toe plate and the support plate, the second force sensor operative to measure a load applied by the four lesser toes to the second toe plate.

13. The device of claim 12, wherein the support plate is movable relative to a ground surface and in unison with the first toe plate and the second toe plate.

14. The device of claim 12, wherein the first toe plate and the second toe plate are pivotally movable about a first axis of rotation between multiple angular positions relative to the ground surface, the first axis of rotation substantially aligned with a first edge of the first toe plate and a first edge of the second toe plate.

15. The device of claim 12, further comprising a motor operable to pivotally move the first toe plate and the second toe plate relative to the ground surface about the first axis of rotation.

16. The device of claim 12, further comprising a rotational position sensor operable to measure an angular position of the first toe plate and the second toe plate relative to the ground surface.

17. The device of claim 12, further comprising at least one wall pivotally supporting the support plate, the first toe plate, and the second toe plate about the first axis of rotation, the at least one wall receiving a retaining member operable to retain the first toe plate and the second toe plate at multiple angles relative to the ground surface.

18. The device of claim 12, further comprising a base plate operable to receive a heel of a foot and defining a longitudinal axis substantially parallel to a ground surface, the base plate extending between a first edge and a second edge with one of the first edge and the second edge being disposed proximate to a first edge of the first toe plate and a first edge of the second toe plate.

19. The device of claim 18, further comprising a heel plate movably secured to the base plate and opposing an upper surface of the base plate, the heel plate including a heel cup operable to receive the heel of the foot.

20. The device of claim 19, wherein the heel plate is linearly movable relative to the base late along the longitudinal axis.

21. The device of claim 19, wherein the heel plate is rotatable relative to the base plate.
